(19) United States
(12) Patent Application Publication
(10) Pub. No.: US 2026/0063740 A1
(43) Pub. Date: Mar. 5, 2026

(54) MEDICAL INFORMATION PROCESSING DEVICE, MEDICAL INFORMATION PROCESSING METHOD, AND MAGNETIC RESONANCE IMAGING APPARATUS

(71) Applicant: CANON MEDICAL SYSTEMS CORPORATION, Otawara-shi (JP)

(72) Inventors: Takuhiro OOSAWA, Yaita (JP); Kaoru IKEDA, Ako (JP); Naoko YAMAGUCHI, Utsunomiya (JP); Keisuke OKUZUMI, Utsunomiya (JP)

(73) Assignee: CANON MEDICAL SYSTEMS CORPORATION, Otawara-shi (JP)

(21) Appl. No.: 19/312,484

(22) Filed: Aug. 28, 2025

(30) Foreign Application Priority Data
Sep. 5, 2024 (JP) 2024-153031

(51) Int. Cl. G01R 33/36; A61B 5/00; A61B 5/055

(52) U.S. Cl. CPC G01R 33/36; A61B 5/0013; A61B 5/055; A61B 5/742

(57) ABSTRACT

According to an embodiment, a medical information processing device for managing a power storage state of a battery provided in a radio frequency (RF) coil unit to be mounted on a subject to be scanned by a magnetic resonance imaging apparatus, the medical information processing device includes processing circuitry. The processing circuitry acquires an imaging condition to be used when the subject is scanned, estimates, based on the imaging condition, an amount of power of the battery of the RF coil unit consumed by a scanning of the subject based on the imaging condition, acquires battery information including an amount of the power stored in the battery, and determines, based on the estimated amount of the power and the acquired battery information, whether or not the RF coil unit can be used to scan the subject based on the imaging condition.

### CROSS-REFERENCE TO RELATED APPLICATION

The present application claims priority based on Japanese Patent Application No. 2024-153031, filed Sep. 5, 2024, the content of which is incorporated herein by reference.

### FIELD

Embodiments of the present invention relate to a medical information processing device, a medical information processing method, and a magnetic resonance imaging apparatus.

### BACKGROUND

Conventionally, magnetic resonance imaging (MRI) apparatuses are used as medical image diagnosis apparatuses that perform image-based diagnosis. A magnetic resonance imaging apparatus (hereinafter referred to as an “MRI apparatus”) is an apparatus that captures a tomographic image of a subject by receiving an MR signal excited by a radio frequency (RF) pulse radiated in a strong magnetic field with an RF coil. In a conventional RF coil, electric power is supplied from a control device via a wire, and control signals and MR signals are exchanged with the control device via a wire. In recent years, a configuration in which the RF coil, which receives MR signals, is wirelessly configured in the MRI apparatus has been considered.

Meanwhile, even if the RF coil is wirelessly configured, it is necessary to supply power to the RF coil that is wirelessly configured (hereinafter referred to as a “wireless RF coil unit”). As a configuration in which power is supplied to the wireless RF coil unit, for example, a battery (a secondary battery) such as a nickel-metal hydride battery can be provided. Although the battery can supply as much power as it stores, it is necessary to charge the battery when the stored power becomes low or runs out. Moreover, because it takes a long time to capture a tomographic image of a subject in an MRI apparatus, there are cases where the battery cannot be sufficiently charged by simply charging the battery between imaging processes. Also, for a performer of an MRI examination (such as a doctor or technician) to perform an examination using a wireless RF coil unit with peace of mind, a method for providing a plurality of wireless RF coil units with fully charged batteries is not necessarily an efficient method. Furthermore, factors that may accelerate the deterioration of the battery may be the iteration of charging and discharging, the charging of the battery in a state in which it has a certain amount of stored power, and the like. For this reason, in an MRI apparatus that receives MR signals using a wireless RF coil unit, it becomes necessary to manage the state of power stored in the battery provided in the wireless RF coil unit, i.e., the battery storage state.

### BRIEF DESCRIPTION OF THE DRAWINGS

FIG. 1 is a block diagram showing an example of a configuration of a medical image diagnosis apparatus including a medical information processing device according to an embodiment.

FIG. 2 is a block diagram showing an example of a functional configuration of the medical information processing device according to the embodiment.

FIG. 3 is a sequence chart showing an example of a timing at which imaging is performed in the medical image diagnosis apparatus including the medical information processing device according to the embodiment.

FIG. 4 is a diagram showing an example of a setting image for setting imaging conditions for an imaging timing in the medical image diagnosis apparatus including the medical information processing device according to the embodiment.

FIG. 5 is a diagram showing an example of a setting image for setting imaging conditions for selecting an RF coil unit in the medical image diagnosis apparatus including the medical information processing device according to the embodiment.

FIG. 6 is a flowchart showing an example of a procedure for imaging a subject in the medical image diagnosis apparatus including the medical information processing device according to the embodiment and a processing flow in the medical information processing device.

### DETAILED DESCRIPTION

According to an embodiment, a medical information processing device for managing a power storage state of a battery provided in an RF coil unit to be mounted on a subject to be scanned by a magnetic resonance imaging apparatus, the RF coil unit being configured to receive a magnetic resonance signal as an analog data from the subject generated by applying a radio frequency (RF) pulse to the subject, to convert the magnetic resonance signal into a magnetic resonance data as a digital data, and to transmit the magnetic resonance data by a wireless communication, the medical information processing device includes processing circuitry. The processing circuitry acquires an imaging condition to be used when the subject is scanned by the magnetic resonance imaging apparatus, estimates, based on the imaging condition, an amount of power of the battery of the RF coil unit consumed by a scanning of the subject based on the imaging condition by the magnetic resonance imaging apparatus, acquires battery information including an amount of the power stored in the battery, and determines, based on the estimated amount of the power and the acquired battery information, whether or not the RF coil unit can be used to scan the subject based on the imaging condition by the magnetic resonance imaging apparatus.

Hereinafter, a medical information processing device, a medical information processing method, and a magnetic resonance imaging apparatus according to embodiments will be described with reference to the drawings. The medical information processing device according to the embodiment is applied to, for example, a magnetic resonance imaging (MRI) apparatus (hereinafter referred to as an “MRI apparatus”).

The MRI apparatus is a medical image diagnosis apparatus that irradiates a subject (e.g., a human body) with RF pulses while applying a strong magnetic field, receives electromagnetic waves generated from hydrogen nuclei in the subject's body by the nuclear magnetic resonance phenomenon with an RF coil, and reconstructs a nuclear magnetic resonance signal (hereinafter referred to as an “MR signal”) based on the received electromagnetic waves to capture a tomographic image (hereinafter referred to as an “MR image”) of the subject. The MRI apparatus can also capture an MR image of the subject by reconstructing an MR signal based on the electromagnetic waves received by an RF coil of a wireless type (hereinafter referred to as a “wireless RF coil unit”) attached to the subject. The MRI apparatus displays the MR image of the subject, thereby allowing the performer of the MRI examination (such as a doctor or technician) to visually ascertain whether or not the subject has a lesion and the like.

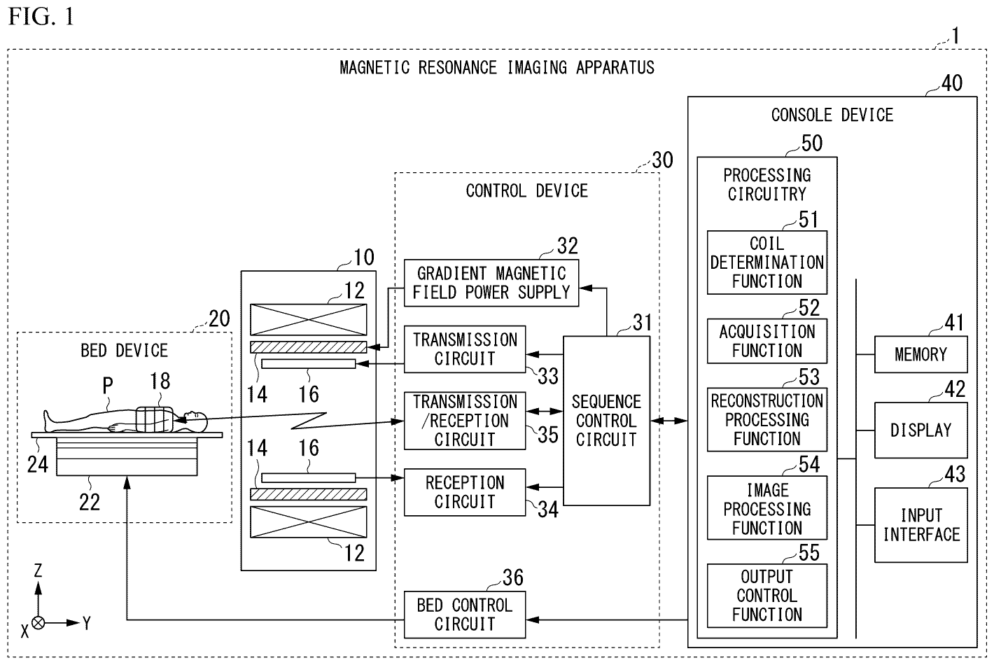

FIG. 1 is a diagram showing an example of the configuration of a medical image diagnosis apparatus (an MRI apparatus) including a medical information processing device according to an embodiment. An MRI apparatus 1 includes, for example, a gantry device 10, a bed device 20, a control device 30, and a console device 40. Although a case where the control device 30 and the console device 40 are separate from the gantry device 10 will be described in the present embodiment, the gantry device 10 may include some or all constituent elements of the control device 30 and the console device 40.

The MRI apparatus 1 is an example of a “medical image diagnosis apparatus.”

The gantry device 10 includes, for example, a static magnetic field magnet 12, a gradient magnetic field coil 14, and an RF coil 16. Furthermore, the gantry device 10 includes, for example, a wireless RF coil unit 18 attachable to a subject P as a constituent element of the RF coil 16.

The static magnetic field magnet 12 is a hollow magnet formed in an approximately cylindrical shape. The static magnetic field magnet 12 generates a uniform static magnetic field in an internal space. The static magnetic field magnet 12 is, for example, a permanent magnet, a superconducting magnet, or the like. When the static magnetic field magnet 12 is a superconducting magnet, it receives power from a static magnetic field power supply (not shown) to generate a static magnetic field.

The gradient magnetic field coil 14 is a hollow coil formed in an approximately cylindrical shape. The gradient magnetic field coil 14 is arranged inside the static magnetic field magnet 12. The gradient magnetic field coil 14 is formed by combining three coils corresponding to axes, i.e., an X-axis, a Y-axis, and a Z-axis, orthogonal to each other. Each of the three coils corresponding to the directions of the axes receives an electric current from the gradient magnetic field power supply 32 individually and generates an inclined magnetic field whose magnetic field strength changes along each of the X-axis, Y-axis, and Z-axis in an imaging space (i.e., in a bore) of the MRI apparatus 1 into which the subject P is introduced. In the present embodiment, a central axis of the gantry device 10 or a longitudinal direction of the top plate 24 of the bed device 20 is defined as a Y-axis direction, an axis orthogonal to the Y-axis direction and horizontal to the floor surface of the room in which the MRI apparatus 1 is installed is defined as an X-axis direction, and a direction orthogonal to the Y-axis direction and perpendicular to the floor surface is defined as a Z-axis direction. Also, in the present embodiment, it is assumed that the Y-axis direction is the same as a direction of the static magnetic field.

Here, the inclined magnetic fields along the X-axis, Y-axis, and Z-axis generated by the gradient magnetic field coil 14, for example, correspond to an inclined magnetic field for slice selection, an inclined magnetic field for phase encoding, and an inclined magnetic field for readout. The inclined magnetic field for slice selection is used to determine any imaging cross-section in the MRI apparatus 1. The inclined magnetic field for phase encoding is used to change the phase of the MR signal in accordance with a spatial position in the MRI apparatus 1. The inclined magnetic field for readout is used to change the frequency of the MR signal in accordance with the spatial position in the MRI apparatus 1.

The RF coil 16 is a whole-body coil that is housed in the gantry device 10 and configured to surround the subject P in the imaging space. The RF coil 16 receives RF pulses from the transmission circuit 33 and generates a high-frequency magnetic field. The RF coil 16 receives MR signals emitted from the subject P due to the influence of the high-frequency magnetic field. When the RF coil 16 receives the MR signals, the received MR signals are output to the reception circuit 34. The RF coil 16 may transmit RF pulses and receive MR signals using different RF coil configurations or using the same RF coil configuration, i.e., a configuration for both transmission and reception. The RF coil 16 may be, for example, a coil array including a plurality of coil elements (a so-called phased-array coil).

The wireless RF coil unit 18 is a wireless local coil attached to the subject P. The wireless RF coil unit 18 is available in various shapes for each site of the subject P to be imaged (hereinafter referred to as each “imaging site”). FIG. 1 shows an example of the wireless RF coil unit 18 attached to the torso of the subject P. The wireless RF coil unit 18 receives an MR signal emitted from the subject P by an influence of a high-frequency magnetic field generated by the RF coil 16 with a coil unit (not shown) and transmits the received MR signal to the transmission/reception circuit 35 by wireless communication. The wireless RF coil unit 18 includes a battery (a secondary battery) such as a nickel-metal hydride battery (not shown) as a power source for each constituent element constituting the wireless RF coil unit 18. Furthermore, the wireless RF coil unit 18 includes a communication interface and a communication antenna (not shown) for wireless communication with the transmission/reception circuit 35. The wireless RF coil unit 18 may also include a coil unit (not shown) of a coil array (a phased-array coil) including a plurality of coil elements. In this case, in the wireless RF coil unit 18, one communication interface (not shown) may sequentially transmit the MR signals received by the coil elements to the transmission/reception circuit 35 by wireless communication or a communication interface (not shown) corresponding to each coil element may sequentially transmit the MR signals received by the corresponding coil elements to the transmission/reception circuit 35 by wireless communication. The wireless RF coil unit 18 may include an analog-to-digital converter (an AD converter) (not shown) that converts the received MR signal (analog signal) into digital value data (hereinafter referred to as “MR data”). In this case, the wireless RF coil unit 18 transmits the MR data after the conversion process of the AD converter (not shown) to the transmission/reception circuit 35 by wireless communication.

The wireless RF coil unit 18 is an example of an “RF coil unit (a wireless RF coil unit).”

The bed device 20 is a device configured to introduce the subject P to be imaged into the inside of the gantry device 10, i.e., into the bore of the gantry device 10, by moving a top plate 24 on which the subject P is placed. In other words, the bed device 20 is a device configured to move the top plate 24 so that the imaging site of the subject P is in a position suitable for imaging within a cavity of the static magnetic field magnet 12, the gradient magnetic field coil 14, and the RF coil 16, i.e., the magnetic field generated within an imaging port. The bed device 20 includes, for example, a base 22 and the top plate 24.

The base 22 moves the top plate 24 on which the subject P is placed in the horizontal direction (the X-axis direction and the Y-axis direction) or the vertical direction (the Z-axis direction) by the operation of a bed drive device (not shown) that operates in accordance with a control signal output from the bed control circuit 36. The base 22 includes a housing that movably supports the top plate 24. The bed drive device (not shown) includes, for example, a motor and an actuator. The bed drive device (not shown) may move not only the top plate 24 but also the base 22 itself in the longitudinal direction (the Y-axis direction) of the top plate 24. When the gantry device 10 is movably configured in the Y-axis direction, the bed drive device (not shown) may move the gantry device 10 so that the subject P is introduced into the gantry device 10. The bed drive device (not shown) may operate so that the subject P is introduced into the gantry device 10 by moving each of the gantry device 10, the top plate 24, and the base 22 in a configuration in which the gantry device 10, the top plate 24, and the base 22 are all movable.

The top plate 24 is a plate-shaped member on which the subject P is placed. The top plate 24 is made of a material with low conductivity (which is less affected by magnetic fields), such as glass fiber.

The control device 30 controls the operations of the gantry device 10 and the bed device 20 in accordance with control from the console device 40. The control device 30 includes, for example, a sequence control circuit 31, a gradient magnetic field power supply 32, a transmission circuit 33, a reception circuit 34, a transmission/reception circuit 35, and a bed control circuit 36. The control device 30 may be provided in the gantry device 10 or in the console device 40.

The sequence control circuit 31 is a sequencer that performs a process of imaging the subject P by driving the gradient magnetic field power supply 32, the transmission circuit 33, the reception circuit 34, and the transmission/reception circuit 35 on the basis of sequence information set by the console device 40. The sequence control circuit 31 may be processing circuitry having a processor such as a central processing unit (CPU). The sequence information is information that defines in advance a procedure for performing an imaging process for imaging the subject P in the MRI apparatus 1. In the sequence information, a procedure is defined in advance for each imaging process performed in the MRI apparatus 1. In the sequence information, for example, operations and operation timings (hereinafter referred to as “events”) of the gradient magnetic field power supply 32, the transmission circuit 33, the reception circuit 34, and the transmission/reception circuit 35 when the subject P is imaged are shown in chronological order. More specifically, in the sequence information, the magnitude of the electric current to be supplied by the gradient magnetic field power supply 32 to the gradient magnetic field coil 14, a timing at which the electric current is supplied, the strength of the RF pulse to be transmitted (supplied) by the transmission circuit 33 to the RF coil 16, a timing at which the RF pulse is supplied, a period during which the RF pulse is supplied, and the like are shown as events. Furthermore, in the sequence information, a timing at which the reception circuit 34 receives (detects) the MR signal output by the RF coil 16, a period during which the MR signal is received (detected), a timing at which the transmission/reception circuit 35 receives (detects) the MR signal (or MR data) output by the wireless RF coil unit 18, a period during which the MR signal is received (detected), and the like are shown as events. The sequence control circuit 31 drives the gradient magnetic field power supply 32, the transmission circuit 33, the reception circuit 34, and the transmission/reception circuit 35 by sequentially executing the events indicated in the sequence information at a timing based on a predetermined clock signal, and transfers the received MR signal (which may be MR data indicating the MR signal) to the console device 40 when the reception circuit 34 or the transmission/reception circuit 35 receives the MR signal. The clock signal, for example, is generated by a clock generation circuit (not shown) including a clock oscillator and indicates a timing that becomes a reference of the operation for imaging the subject P in the MRI apparatus 1. The clock signal is supplied to each of the constituent elements provided in the control device 30. The sequence control circuit 31 executes events in sequence at timing based on the clock signal, such that the gradient magnetic field power supply 32, the transmission circuit 33, and the reception circuit 34 operate in synchronization with each other. Furthermore, the sequence control circuit 31 causes the transmission/reception circuit 35 to transmit the clock signal and data representing an event for driving the wireless RF coil unit 18 (hereinafter referred to as “event data”). The clock signal is a clock signal used (as a reference) when the sequence control circuit 31 drives the gradient magnetic field power supply 32, the transmission circuit 33, the reception circuit 34, and the transmission/reception circuit 35, and the wireless RF coil unit 18 operates on the basis of the transmitted clock signal, such that the wireless RF coil unit 18 also operates in synchronization with the gradient magnetic field power supply 32, the transmission circuit 33, and the reception circuit 34.

The gradient magnetic field power supply 32 supplies an electric current individually to each of the three coils corresponding to each axial direction in the gradient magnetic field coil 14.

The transmission circuit 33 supplies an RF pulse to the RF coil 16. The RF pulse supplied by the transmission circuit 33 to the RF coil 16 is a pulse corresponding to a Larmor frequency determined by a type of atomic nucleus of interest and the strength of the magnetic field.

The reception circuit 34 detects the MR signal output by the RF coil 16 and generates MR data indicating the detected MR signal. The reception circuit 34, for example, generates the MR data by converting the MR signal into digital data. The reception circuit 34 outputs the generated MR data to the sequence control circuit 31. The sequence control circuit 31 transfers the MR data output by the reception circuit 34 to the console device 40.

The transmission/reception circuit 35 transmits a clock signal and event data to the wireless RF coil unit 18 in accordance with control from the sequence control circuit 31. The transmission/reception circuit 35 receives the MR signal transmitted by the wireless RF coil unit 18. The transmission/reception circuit 35, for example, generates MR data by converting the MR signal into digital data. When the wireless RF coil unit 18 is configured to transmit MR data, the transmission/reception circuit 35 receives the MR data transmitted by the wireless RF coil unit 18. The transmission/reception circuit 35 performs the transmission of the clock signal and event data and the reception of the MR signal and MR data using, for example, a wireless communication standard such as Wi-Fi. The transmission/reception circuit 35 includes, for example, an antenna (not shown) corresponding to the wireless communication standard. The transmission/reception circuit 35 outputs the generated or received MR data to the sequence control circuit 31.

The bed control circuit 36 outputs a control signal for moving the base 22 and the top plate 24 on which the subject P is placed to a bed drive device (not shown) provided in the bed device 20 in accordance with control from the console device 40. The bed control circuit 36 may be provided in the gantry device 10 or in the bed device 20. In this case, the bed control circuit 36 outputs a control signal to the bed drive device (not shown) provided in the bed device 20 according to an input signal input from an input interface (not shown) by an operator of the MRI apparatus 1 such as a doctor or technician or the performer of the MRI examination (hereinafter referred to as the “performer of the MRI examination”) operating the input interface (not shown) provided in the device in which the bed control circuit 36 is provided.

The console device 40 controls the entire MRI apparatus 1 and collects MR data. The console device 40 includes, for example, a memory 41, a display 42, an input interface 43, and processing circuitry 50.

The memory 41, for example, is implemented by a semiconductor memory element such as a read only memory (ROM), a random-access memory (RAM), or a flash memory, a hard disk drive (HDD), an optical disk, or the like. The memory 41, for example, stores data such as MR data output by the sequence control circuit 31 and a reconstructed image (an MRI image) generated on the basis of the MR data. The data may be stored in an external memory with which the MRI apparatus 1 can communicate, instead of the memory 41 (or in addition to the memory 41). The external memory, for example, is controlled by a cloud server that manages a network attached storage (NAS) or the external memory when the cloud server receives a read/write request. The external memory is, for example, implemented by a system referred to as a picture archiving and communication system (PACS). The PACS is a medical image management system that systematically stores medical images captured by various types of medical image diagnosis apparatuses and the like.

The display 42 displays various types of information. For example, the display 42 displays medical images generated by the processing circuitry 50, graphical user interface (GUI) images for receiving various types of operations from the performer of the MRI examination, and the like. The display 42 is, for example, a liquid crystal display (LCD), a cathode ray tube (CRT) display, an organic electroluminescence (EL) display, or the like. The display 42 may be provided in the gantry device 10. The display 42 may be a desktop type or may be a display device (e.g., a tablet terminal) that can wirelessly communicate with the main body of the console device 40.

The input interface 43 receives various types of input operations from the performer of the MRI examination and outputs an electrical signal indicating the content of the received input operation to the processing circuitry 50. For example, the input interface 43 receives operations for inputting a collection condition when MR data is collected (i.e., an imaging condition when the subject P is imaged), a generation condition when the MR data is generated, a reconstruction condition when a reconstructed image is reconstructed, an image processing condition when a post-processed image is generated from the reconstructed image, and the like. The input interface 43, for example, is implemented by a mouse, a keyboard, a touch panel, a trackball, a switch, a button, a joystick, a camera, an infrared sensor, a microphone, and the like. When the input interface 43 is a touch panel, the display 42 may be formed integrally with the input interface 43. The input interface 43 may be provided in the gantry device 10. The input interface 43 may be implemented by a display device (e.g., a tablet terminal) capable of wireless communication with the main body of the console device 40. In the present specification, the input interface 43 is not limited to only those having physical operation parts such as the mouse and keyboard described above. For example, an electrical signal processing circuit that receives an electrical signal corresponding to an input operation from an external input device provided separately from the console device 40 and outputs this electrical signal to the processing circuitry 50 is also included in an example of the input interface 43.

The processing circuitry 50 controls the overall operation of the MRI apparatus 1. The processing circuitry 50 sets sequence information in the sequence control circuit 31. The processing circuitry 50, for example, executes a coil determination function 51, an acquisition function 52, a reconstruction processing function 53, an image processing function 54, an output control function 55, and the like. The processing circuitry 50, for example, implements these functions with a hardware processor provided in a computer device executing a program (software) stored in the memory 41, which is a storage device (a storage circuit).

The hardware processor is, for example, circuitry such as a CPU, a graphics processing unit (GPU), a large-scale integration (LSI) circuit, a system on chip (SOC), an application-specific integrated circuit (ASIC), or a programmable logic device (e.g., a simple programmable logic device (SPLD), a complex programmable logic device (CPLD), or a field programmable gate array (FPGA)). Instead of storing a program in the memory 41, the program may be directly built into the circuitry of the hardware processor. In this case, the hardware processor implements a function by reading and executing the program built into the circuitry. The hardware processor is not limited to a configuration as a single circuit and may be configured as one hardware processor by combining a plurality of independent circuits to implement each function. A plurality of constituent elements may be integrated into one hardware processor to implement each function. A plurality of constituent elements may be built into one dedicated LSI circuit to implement each function. Here, the program (software) may be stored in advance in a storage device (a storage device having a non-transitory storage medium) constituting the memory 41 such as a semiconductor memory element such as a ROM, a RAM, or a flash memory or a hard disk drive (HDD). Alternatively, the program may be stored in a removable storage medium (a non-transitory storage medium) such as a DVD or CD-ROM and installed in the storage device provided in the console device 40 when the storage medium is mounted in a drive device provided in the console device 40. The program (software) may be downloaded in advance from another computer device via a network (not shown) and installed in the storage device provided in the console device 40.

The constituent elements of the console device 40 or the processing circuitry 50 may be distributed and implemented by a plurality of pieces of hardware. The processing circuitry 50 may be implemented by a processing device capable of communicating with the console device 40, instead of a configuration provided in the console device 40. The processing device is, for example, a workstation connected to one MRI apparatus or a device (e.g., a cloud server) connected to a plurality of MRI apparatuses and configured to collectively execute processes equivalent to those of the processing circuitry 50 to be described below. In other words, the configuration of the present embodiment can also be implemented as an MRI examination system (a medical diagnostic system) in which an MRI apparatus and another processing device are connected via a network.

Because the power storage state of the battery provided in the wireless RF coil unit 18 is managed, when an MR image of the subject Pis captured using the wireless RF coil unit 18 in the MRI apparatus 1, the coil determination function 51 is executed to determine whether or not imaging can be performed without problems using the wireless RF coil unit 18 by estimating the power usage capacity of the battery provided in the wireless RF coil unit 18, i.e., the battery power consumption during imaging. More specifically, the coil determination function 51, for example, is executed to estimate the power consumption of the battery included in the wireless RF coil unit 18 on the basis of an imaging condition when the subject P is imaged and information about the battery provided in the wireless RF coil unit 18 (hereinafter referred to as “battery information”). It is determined whether or not imaging using the wireless RF coil unit 18 can be performed by comparing the estimated battery power consumption with at least the capacity of power currently stored in the battery, in other words, the remaining amount of power of the battery. The coil determination function 51 is executed to present (show) a result of determining whether or not imaging using the wireless RF coil unit 18 can be performed (a determination result) to the performer of the MRI examination. When the determination result indicates that imaging using the wireless RF coil unit 18 cannot be performed, the coil determination function 51 may be executed to propose a change in the imaging using the wireless RF coil unit 18.

The coil determination function 51 is an example of a “medical information processing device.”

The acquisition function 52 is executed to acquire the MR data transferred by the sequence control circuit 31. The MR data is obtained by converting an MR signal into digital data in the reception circuit 34 or is received by the transmission/reception circuit 35 from the wireless RF coil unit 18. The acquisition function 52 may cause the memory 41 to store the MR data that has been acquired.

The reconstruction processing function 53 is executed to generate a reconstructed image by performing a predetermined reconstruction process on the MR data (which may be the MR data stored in the memory 41) acquired by the acquisition function 52. For example, the reconstruction processing function 53 is executed to arrange the MR data in two dimensions or three dimensions corresponding to an inclined magnetic field for slice selection, an inclined magnetic field for phase encoding, and an inclined magnetic field for readout and then perform a reconstruction process using a Fourier transform or the like to generate the reconstructed image. The reconstruction processing function 53 causes the memory 41 to store the generated reconstructed image.

The image processing function 54 is executed to perform predetermined image processing on the reconstructed image stored in the memory 41 on the basis of the input operation received by the input interface 43 and generate an MR image to be presented to the performer of the MRI examination. The predetermined image processing is, for example, a process of converting the reconstructed image into a three-dimensional image or cross-sectional image data of any cross-section by a known method. The image processing function 54 causes the memory 41 to store the generated MR image.

The output control function 55, for example, is executed to control a display aspect in the display 42. The output control function 55 is executed to output a determination result of the coil determination function 51 to the display 42 so that the determination result is displayed. Thereby, the performer of the MRI examination can change the imaging condition or the sequential order in which imaging is performed as necessary on the basis of the determination result displayed on the display 42. The output control function 55 is executed to output an MR image generated by the image processing function 54 and stored in the memory 41 to the display 42 so that the MR image is displayed. Thereby, the performer of the MRI examination can visually ascertain the MR image displayed on the display 42 and perform the diagnosis or examination of whether or not the subject P has a lesion or the like. The output control function 55 may be executed to transmit the MR image to, for example, a tablet terminal connected to a main body of the console device 40 via a network (not shown), and cause a display device to display the MR image. The output control function 55 may be executed so that a GUI image for receiving various types of operations from the performer of the MRI examination and the like are displayed.

### [Configuration and Operation of Medical Information Processing Device]

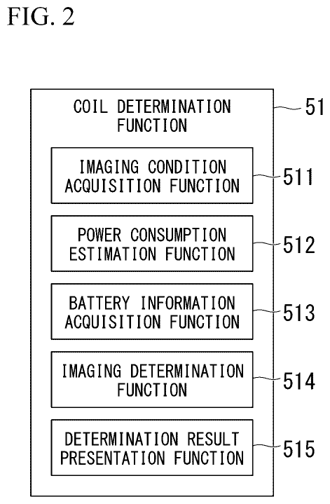

Next, a configuration and operation of the coil determination function 51 will be described. FIG. 2 is a diagram showing an example of the functional configuration of the medical information processing device (the coil determination function 51) according to the embodiment. For example, an imaging condition acquisition function 511, a power consumption estimation function 512, a battery information acquisition function 513, an imaging determination function 514, a determination result presentation function 515, and the like are executed by the coil determination function 51.

The imaging condition acquisition function 511 is executed to acquire imaging conditions for use in estimating the power consumption of the battery provided in the wireless RF coil unit 18. The imaging condition acquisition function 511, for example, is executed to acquire the imaging conditions set in the MRI apparatus 1 by the performer of the MRI examination so that the subject P is imaged.

The main power consumption of the battery provided in the wireless RF coil unit 18, for example, can be calculated using a usage time obtained by combining a standby time and an operation time of a coil unit (not shown), i.e., a control time of the coil unit. The main operation time of the coil unit includes, for example, a reception time of the MR signal and the time required for the transfer (transmission) of the received MR signal. The reception time of the MR signal, for example, can be calculated using the time per reception of the MR signal, the number of receptions, and the number of coil units receiving the MR signal. The transfer time of the MR signal, for example, varies with the magnitude of the MR signal to be transmitted (an amount of MR data when the wireless RF coil unit 18 is configured to transmit MR data). In other words, the transfer time of the MR signal is the time of wireless communication in the communication interface (not shown). The magnitude of the MR signal and the amount of MR data are proportional to the strength of the high-frequency magnetic field generated when imaging is performed, the strength of the RF pulse, the period during which the RF pulse is supplied, and the like.

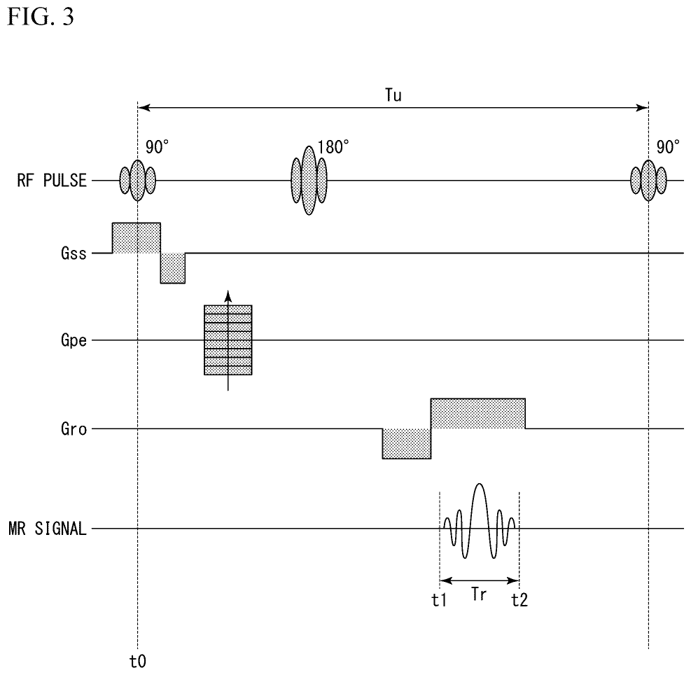

Here, an example of the reception time of the MR signal will be described. FIG. 3 is a sequence chart showing an example of a timing at which imaging is performed in a medical image diagnosis apparatus (the MRI apparatus 1) including a medical information processing device (the coil determination function 51) according to the embodiment. In FIG. 3, an example of a reception time period (hereinafter referred to as a “reception period Tr”) during which the wireless RF coil unit 18 receives MR signals in a certain period (hereinafter referred to as a “unit period Tu”) during which the sequence control circuit 31 executes events in sequential order to receive (detect) MR signals for one reception (detection) process is shown. More specifically, in FIG. 3, an example of time relationships between RF pulses schematically indicating an example of a state in which the RF pulses are supplied from the transmission circuit 33 and are actually radiated within the unit period Tu, each of an inclined magnetic field Gss for slice selection, an inclined magnetic field Gpe for phase encoding, and an inclined magnetic field Gro for readout generated by the gradient magnetic field coil 14, an MR signal schematically indicating an example of a state in which the MR signal is emitted from the subject P, and a reception period Tr is shown. In the example shown in FIG. 3, after the RF coil 16 radiates an RF pulse at time to, the wireless RF coil unit 18 receives an MR signal emitted from the subject P during the MR signal reception period Tr (a period from time t1 to time t2).

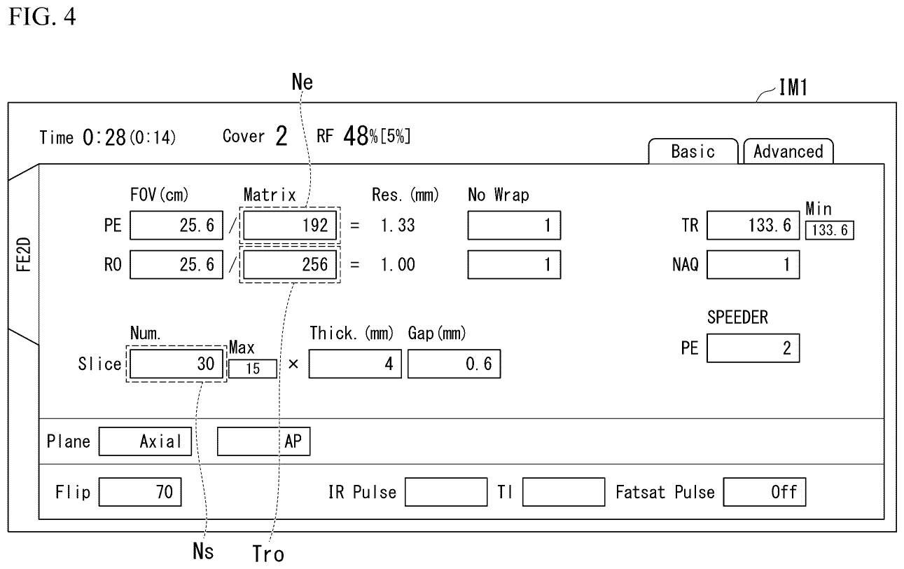

The performer of the MRI examination operates the input interface 43 provided in the console device 40 to set an imaging condition for imaging the subject P at the timing of the example shown in FIG. 3 on a setting image (a GUI image) for setting the imaging condition displayed on the display 42 in the MRI apparatus 1. The performer of the MRI examination, for example, sets the number of selections (the number of slices) based on the inclined magnetic field Gss for slice selection in the unit period Tu, the number of times (the number of encodings) the inclined magnetic field Gpe for phase encoding is repeatedly generated in the gradient magnetic field coil 14, the timing of the reception period Tr, and the like. FIG. 4 is a diagram showing an example of a setting image for setting an imaging condition of an imaging timing in the medical image diagnosis apparatus (the MRI apparatus 1) including the medical information processing device (the coil determination function 51) according to the embodiment. In the example of the setting image IM1 shown in FIG. 4, an example in which the number of slices Ns=30, the number of encodings Ne=192, and a timing Tro of the reception period Tr=256 are set is shown. The setting image for setting the imaging conditions is not limited to the example such as the setting image IM1 shown in FIG. 4. The setting image may include items for setting various imaging conditions including an item for setting the number of MR images to be averaged when another MR image is generated by averaging the captured MR images and the like. The imaging condition acquisition function 511 is executed to acquire information (setting values) set by the performer of the MRI examination as imaging conditions.

Although an example of the timing of a reception period Tr in which the wireless RF coil unit 18 receives MR signals in a unit period Tu for receiving (detecting) MR signals for one reception (detection) process is shown in an example shown in FIG. 3, the irradiation of RF pulses and the reception of MR signals are performed a plurality of times when the subject P is imaged in the MRI apparatus 1. Therefore, the imaging condition acquisition function 511 is executed to acquire information (a setting value) on the imaging timing corresponding to each unit period Tu set by the performer of the MRI examination as each imaging condition.

Furthermore, a configuration in which the coil unit of the wireless RF coil unit 18 is a coil array including a plurality of coil elements, and the coil elements not used for imaging are not affected by the high-frequency magnetic field generated by the RF coil 16 is conceivable. For example, a case where the coil unit and coil elements may be configured to be disconnected by a high-frequency diode (a so-called PIN diode) so that they do not function as an RF coil normally and to be connected so that they function as an RF coil only when used for imaging (receiving MR signals) is also conceivable to prevent the coil unit and coil elements from being heated or destroyed by eddy currents generated by the high-frequency magnetic field. In this case, the power consumption of the battery by the high-frequency diode to function as an RF coil is also included in the power consumption of the battery provided in the wireless RF coil unit 18.

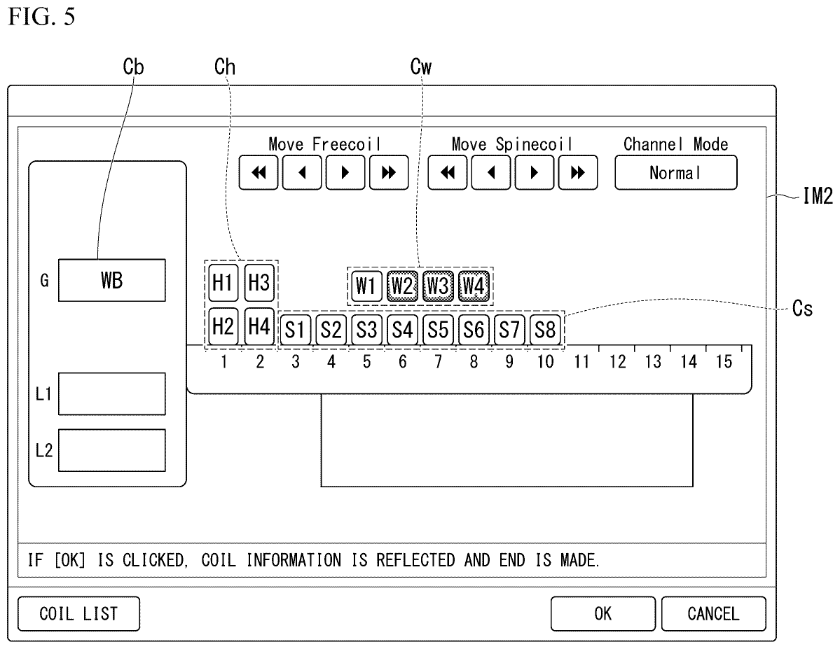

The performer of the MRI examination operates the input interface 43 provided in the console device 40 to set imaging conditions for designating an RF coil that receives an MR signal when the subject P is imaged and a coil element within a coil array in the MRI apparatus 1 on a setting image (a GUI image) for selecting an RF coil displayed on the display 42. FIG. 5 is a diagram showing an example of a setting image for setting imaging conditions for selecting an RF coil in a medical image diagnosis apparatus (the MRI apparatus 1) including a medical information processing device (the coil determination function 51) according to an embodiment. In FIG. 5, the gantry device 10 and the bed device 20 constituting the MRI apparatus 1 are schematically shown and the RF coil and the coil element that can be used for imaging the subject P in the MRI apparatus 1 are schematically shown. In the example shown in FIG. 5, a state in which an RF coil Cb (i.e., the RF coil 16) in the gantry device 10, a head RF coil Ch (the coil elements H1 to H4) in a coil array configuration in which the head of the subject P can be imaged, a spine RF coil Cs (coil elements S1 to S8) in a coil array configuration in which the spine of the subject P can be imaged, and a wireless RF coil Cw (e.g., the wireless RF coil unit 18: the coil elements W1 to W4) capable of being attached to the subject P can be used for imaging the subject P is schematically shown. Although a case where power is supplied via the top plate 24 because the head RF coil Ch and the spine RF coil Cs are RF coils installed on the top plate 24 is conceivable, one or both of the head RF coil Ch and the spine RF coil Cs may be wireless RF coil units configured so that power is supplied from a battery (not shown) like the RF coil Cw (the wireless RF coil unit 18). In the example shown in FIG. 5, for example, when an RF coil capable of being used for imaging the subject P is added or replaced with a different one, it is only necessary for the corresponding RF coil to be displayed in a setting image IM2 as an RF coil that can currently be used for imaging the subject P. The performer of the MRI examination selects (designates) one or more RF coils or coil elements shown in FIG. 5, and hence the MRI apparatus 1 performs imaging of the subject P using the selected (designated) RF coil. In the example shown in FIG. 5, a state in which the coil elements W2, W3, and W4 in the coil array constituting the wireless RF coil Cw are selected (designated) by the performer of the MRI examination is shown. The imaging condition acquisition function 511 is also executed to acquire information about the RF coils and coil elements selected (designated) by the performer of the MRI examination as imaging conditions. The imaging conditions include, for example, information for identifying the selected (designated) RF coils and coil elements and information indicating the number of RF coils and the number of coil elements (in other words, information indicating the type of RF coil used for imaging, the number of channels of the coil elements, and the like). In the MRI apparatus 1, the imaging conditions of the imaging timing described using FIGS. 3 and 4 can be set for each RF coil. In this case, the imaging condition acquisition function 511, for example, is executed to acquire information (a setting value) of each imaging timing corresponding to each RF coil designated in the setting image IM2 as the imaging condition.

The imaging conditions acquired by the imaging condition acquisition function 511 are not limited to the above-described imaging timing information (setting values) or information about the RF coils and coil elements designated for use in imaging the subject P, and may be any information that can be used to estimate the power consumption of the battery provided in the wireless RF coil unit 18. In addition to the above-described imaging conditions, for example, the imaging condition acquisition function 511 may be executed to acquire information including information about the physique of the subject P (hereinafter referred to as “subject information”), information about the imaging site (hereinafter referred to as “imaging site information”), and the like. The subject information is, for example, information such as the height and weight of the subject P to be imaged. For example, the information about the height of the subject P can be used as information for assuming the RF coils and coil elements for use in imaging when the RF coils and coil elements are not selected (designated) by the performer of the MRI examination. The imaging site information is, for example, information associated with one or more sequence information items predetermined for each imaging site, i.e., a series of sequence information items for each imaging site of the subject P to be imaged. In other words, the imaging site information is information from which the above-described imaging timing information (setting values) and information about the RF coils and coil elements used for imaging, which are preset, can be obtained. The imaging site information, for example, may be predetermined during the development, design, and manufacture of the MRI apparatus 1 or may be predetermined in a medical institution in which the MRI apparatus 1 is installed. The imaging site information, for example, may be stored in the memory 41 or in an external memory with which the MRI apparatus 1 whose reading and writing are controlled by a NAS, a PACS, or a cloud server can communicate.

The imaging condition acquisition function 511 is executed to output information about the acquired imaging conditions (hereinafter referred to as “imaging condition information”) to the power consumption estimation function 512. The imaging condition acquisition function 511 may be executed to cause the memory 41 to store the acquired information about the imaging conditions (imaging condition information) and notify the power consumption estimation function 512 of the information.

The imaging condition acquisition function 511 is an example of an “imaging condition acquirer.”

Returning to FIG. 2, the power consumption estimation function 512 is executed to calculate the power consumption of the battery provided in the wireless RF coil unit 18 for use in imaging on the basis of the imaging condition information output by the imaging condition acquisition function 511. In other words, the power consumption estimation function 512 is executed to estimate the power consumption of the battery for use in imaging on the basis of the imaging condition information. As described above, the imaging condition information output by the imaging condition acquisition function 511 is information (setting values) about an imaging timing at which the subject Pis imaged in the MRI apparatus 1 and information about the RF coil and coil element to be used. In other words, the imaging condition information is information related to the reception of MR signals in the wireless RF coil unit 18. For this reason, the power consumption estimation function 512 is executed to calculate the power consumption of the battery when the wireless RF coil unit 18 receives MR signals on the basis of the imaging condition information. An amount of power of the battery when the wireless RF coil unit 18 receives an MR signal can be considered to be the magnitude of the MR signal received by the wireless RF coil unit 18 (the amount of MR data when the wireless RF coil unit 18 is configured to transmit MR data), and is proportional to an amount of transmission power when the wireless RF coil unit 18 transfers (transmits) the MR signal (or MR data) to the transmission/reception circuit 35 by wireless communication. The power consumption estimation function 512 is executed to set the calculated amount of power of the battery when the MR signal is received as the estimated power consumption of the battery.

When a plurality of wireless RF coil units 18 are used in imaging the subject P, the power consumption estimation function 512 is executed to estimate the power consumption of the battery for each wireless RF coil unit 18. Furthermore, the power consumption estimation function 512 is executed to estimate the power consumption of the battery for each sequence information item. More specifically, the power consumption estimation function 512 is executed to estimate the power consumption of the battery for each sequence information item shown in the imaging condition information, for each sequence information item included in the imaging site information (a series of sequence information items corresponding to the same imaging site), and for each sequence information item included in the series of sequence information items corresponding to the same subject P. Furthermore, the power consumption estimation function 512 is executed to estimate the power consumption of the battery for a series of sequence information items. More specifically, the power consumption estimation function 512 is executed to estimate the power consumption of the battery for each imaging site for which a series of sequence information items in the same imaging site information are combined or in units of diagnosis or examination in which a series of sequence information items corresponding to the same subject P are combined.

The power consumption estimation function 512 is executed to output information about the estimated power consumption of the battery provided in each wireless RF coil unit 18 (hereinafter referred to as “power consumption information”) to the imaging determination function 514. The power consumption estimation function 512 may be executed to cause the memory 41 to store the estimated information about the power consumption of each battery (power consumption information) and notify the imaging determination function 514 of the information.

The power consumption estimation function 512 is an example of a “power consumption estimator.”

The battery information acquisition function 513 acquires battery information of the battery provided in the wireless RF coil unit 18. The battery information includes information about a deterioration state of the battery and the like in addition to information about the power capacity such as a total power capacity of the battery provided in the wireless RF coil unit 18 or a currently stored power capacity. The battery information may be transmitted from the wireless RF coil unit 18, i.e., from a communication interface (not shown) included in the wireless RF coil unit 18, by wireless communication or may be transmitted from a communication interface (not shown) included in a charger that charges the battery of the wireless RF coil unit 18 (which may be wired or wireless). The battery information may be transmitted periodically at a predetermined time interval, or may be transmitted at any timing. For example, when the battery information is acquired from the wireless RF coil unit 18, a communication interface (not shown) provided in the wireless RF coil unit 18 may transmit current battery information by wireless communication at any timing after the imaging of the subject P is completed, and the battery information acquisition function 513 may be executed to acquire this battery information. For example, when the battery information is acquired from a charger that charges the battery of the wireless RF coil unit 18, the charger may periodically transmit current battery information at a predetermined time interval while the battery is being charged, and the battery information acquisition function 513 may be executed to acquire this battery information. The configuration of the charger and the method for charging the battery in the charger are not particularly specified. The charger may be a charger that starts charging the battery when it is connected to an RF coil installed on the top plate 24 or the wireless RF coil unit 18 placed on the top plate 24, such as the head RF coil Ch or the spine RF coil Cs shown in FIG. 5 or a charger that starts charging the battery when the wireless RF coil unit 18 is stored in a coil rack that stores a plurality of wireless RF coils. In the case of the coil rack, instead of the charger, a communication interface (not shown) provided in the main body of the coil rack may transmit battery information of each battery provided in each stored wireless RF coil unit (the wireless RF coil unit 18, the wireless head RF coil Ch, and the spine RF coil Cs (see FIG. 5)), and the battery information acquisition function 513 may be executed to acquire this battery information. The battery information acquisition function 513 may be executed to store the acquired battery information in the memory 41 and read the battery information from the memory 41 at a necessary timing. In this case, when the battery information acquisition function 513 is executed to acquire new battery information corresponding to the same battery, the battery information corresponding to the same battery stored in the memory 41 is updated to new battery information. In other words, the battery information acquisition function 513 is executed to constantly set the battery information corresponding to the same battery stored in the memory 41 to the latest information.

The battery information acquisition function 513 is executed to output the acquired battery information (which may be battery information read from the memory 41) to the imaging determination function 514. The battery information acquisition function 513 may be executed to directly acquire the transmitted battery information and output the transmitted battery information to the imaging determination function 514. When the acquired battery information is stored in the memory 41, the battery information acquisition function 513 may be executed to notify the imaging determination function 514 of a storage area of the memory 41 where the battery information to be output is stored.

The battery information acquisition function 513 is an example of a “battery information acquirer.”

The imaging determination function 514 is executed to determine whether or not imaging using the wireless RF coil unit 18 can be performed on the basis of the power consumption information output by the power consumption estimation function 512 and the battery information output by the battery information acquisition function 513. More specifically, the imaging determination function 514 is executed to compare the power consumption information with the currently stored power capacity indicated in the battery information, determine that imaging using the wireless RF coil unit 18 can be performed without problems when the current power capacity is greater than the power consumption indicated in the power consumption information, and determine that imaging using the wireless RF coil unit 18 cannot be performed without problems when the current power capacity is less than or equal to the power consumption indicated in the power consumption information. The imaging determination function 514 is executed to determine whether or not imaging using the wireless RF coil unit 18 can be performed for each power consumption indicated in the power consumption information output by the power consumption estimation function 512. In other words, the imaging determination function 514 is executed to determine whether or not imaging using the wireless RF coil unit 18 can be performed for the power consumption indicated in the power consumption information which is a combination of a series of sequence information items output by the power consumption estimation function 512, and determine whether or not imaging using the wireless RF coil unit 18 can be performed for power consumption indicated in each power consumption information item output by the power consumption estimation function 512, i.e., for each wireless RF coil unit 18, for each sequence information item, for each imaging site, and for each unit of diagnosis or examination.

When it is determined whether or not imaging using the wireless RF coil unit 18 can be performed, the imaging determination function 514 is executed to make the determination under a determination condition in which a predetermined margin is provided for either the power consumption of the battery indicated in the power consumption information or the current power capacity. More specifically, the imaging determination function 514 is executed to determine whether or not imaging can be performed under a determination condition in which the power consumption of the battery indicated in the power consumption information is higher by the predetermined margin. Alternatively, the imaging determination function 514 is executed to determine whether or not imaging can be performed under a determination condition in which the current power capacity is lower by the predetermined margin. The predetermined margin may be a predetermined ratio (e.g., 10%) or may be decided in accordance with a power capacity that can be used for imaging even if reimaging is required (e.g., a power capacity that enables at least the same imaging to be performed twice, i.e., a power capacity that can be used for reimaging, or the like) or the power consumption of the battery.

When it is determined whether or not imaging using the wireless RF coil unit 18 can be performed in a step of reserving imaging for the subject P (i.e., in a state in which imaging is not currently being performed), the imaging determination function 514 is executed to determine whether or not imaging can be performed under the assumption that the batteries of all the wireless RF coil units 18 are fully charged, instead of the current battery information output by the battery information acquisition function 513.

The imaging determination function 514 is executed to output each of results of determining whether or not imaging using the wireless RF coil unit 18 can be performed for the power consumption indicated in each power consumption information item to the determination result presentation function 515. The imaging determination function 514 may be executed to cause the memory 41 to store each determination result and notify the determination result presentation function 515 of the determination result.

The imaging determination function 514 is an example of an “imaging determiner.”

The determination result presentation function 515 is executed to present (show) the determination result output by the imaging determination function 514 to the performer of the MRI examination. More specifically, the determination result presentation function 515 is executed to present (show) a determination result of a state having a margin corresponding to the estimated battery power consumption in which a series of sequence information items are combined to the performer of the MRI examination. The determination result presentation function 515, for example, is executed to present (show) whether or not imaging using the wireless RF coil unit 18 can be performed without problems to the performer of the MRI examination by generating a determination result image indicating the determination result, outputting the generated determination result image to the output control function 55, and causing the display 42 to display the determination result.

When the determination result output by the imaging determination function 514 is a result indicating that imaging using the wireless RF coil unit 18 cannot be performed without problems, the determination result presentation function 515 may be executed to propose a change in imaging using the wireless RF coil unit 18 to the performer of the MRI examination. That is, even if the determination result of the state having the margin corresponding to the estimated battery power consumption in which the series of sequence information items are combined indicates that imaging using the wireless RF coil unit 18 cannot be performed, when imaging using the wireless RF coil unit 18 can be performed by changing the imaging condition or the determination condition, the determination result presentation function 515 may be executed to present (show) the fact that imaging can be performed if the imaging condition and the determination condition are changed to the performer of the MRI examination. In this case, the determination result presentation function 515 is executed to propose a change in the imaging condition or the determination condition (a change in imaging using the wireless RF coil unit 18) to the performer of the MRI examination on the basis of each determination result for power consumption indicated in each power consumption information item output by the imaging determination function 514. As proposals to change imaging using the wireless RF coil unit 18, for example, the following proposals are conceivable.
(Proposal 1): The determination result presentation function 515 is executed to propose a process for changing (reducing or eliminating) the margin, which is a determination condition when it is determined whether or not imaging using the wireless RF coil unit 18 can be performed. If proposal 1 is received, the performer of the MRI examination can determine whether or not to perform imaging using the wireless RF coil unit 18 after recognizing that there is no margin in the capacity of power currently stored in the battery.
(Proposal 2): The determination result presentation function 515 is executed to propose a change in the imaging conditions. Proposal 2 includes, for example, a proposal to change the number of slices Ns, the number of encodings Ne, and the timing Tro of the reception period Tr set in the example of the setting image IM1 shown in FIG. 4, a proposal to change the selection (designation) of the RF coil and coil element set in the example of the setting image IM2 shown in FIG. 5, and the like. Proposal 2 may include, for example, a proposal to change the effective field of view (FOV) when imaging is performed. If proposal 2 is received, the performer of the MRI examination can determine whether or not an imaging condition to be set again is allowable for diagnosing and examining the subject P in consideration of the capacity of power currently stored in the battery.
(Proposal 3): The determination result presentation function 515 is executed to propose a process for omitting sequence information for capturing the MR images with low clinical significance or changing the sequential order of capture, according to whether or not imaging based on N^{th} sequence information (where N is a natural number) can be performed in imaging based on the series of sequence information items. In other words, the determination result presentation function 515, for example, is executed to make a proposal not to capture MR images with low importance or to postpone the capture of MR images with low importance under the assumption that MR images to be captured on the basis of a series of sequence information items corresponding to an imaging site or MR images to be captured on the basis of a series of sequence information items for diagnosing or examining the subject P cannot be captured midway. If a proposal 3 is received, the performer of the MRI examination can determine whether or not the capture of MR images that can be captured with the capacity of power currently stored in the battery is allowable for diagnosing or examining the subject P.
(Proposal 4): The determination result presentation function 515 is executed to propose imaging using another wireless RF coil unit 18 that can capture MR images similar to those of the selected (designated) wireless RF coil unit 18. The determination result presentation function 515, for example, is executed to propose the use of the same type of wireless RF coil unit 18 with a different size as a substitute on the basis of subject information of the subject P. For example, in a case where a plurality of wireless RF coil units 18 of the same type are stored such as a case where there are a plurality of wireless RF coil units 18 of the same type and size stored in a coil rack, the determination result presentation function 515 is executed to propose the use of another wireless RF coil unit 18 having a large capacity of currently stored battery power different from the wireless RF coil unit 18 currently prepared (or removed from the coil rack). If proposal 4 is received, the performer of the MRI examination can determine whether or not imaging can be performed using a substitute or another wireless RF coil unit 18. Also, when the performer of the MRI examination determines to perform imaging using an alternative wireless RF coil unit 18 or another wireless RF coil unit 18, the selected (designated) wireless RF coil unit 18 is connected to a charger or stored in the coil rack, thereby charging the battery.
(Proposal 5): When the determination result is presented (shown) in a step in which imaging of the subject P is reserved, the determination result presentation function 515 is executed to make a proposal to replace the sequential order of the subject P with the sequential order of another subject who is already scheduled to be imaged on the same day in the reservation. Proposal 5 is a proposal to prevent the capacity of power stored in the battery including a specific wireless RF coil unit 18 from becoming extremely low by continuously performing imaging using the same wireless RF coil unit 18. The determination result presentation function 515 may be executed to present (show) a warning indicating that the capacity of power stored in the battery is low to the performer of the MRI examination in addition to or instead of proposal 5 under the assumption that imaging using the same wireless RF coil unit 18 cannot be continuously performed after the currently reserved imaging of the subject P is completed. If proposal 5 (or the above-described warning) is received, the performer of the MRI examination can make a reservation for imaging the subject P in consideration of the rearrangement of an imaging plan for the same reservation date, whether the reservation for the subject P can be made on another day, and the like.

The determination result presentation function 515 is an example of a “determination result presenter.”

According to such a configuration and operation, the coil determination function 51, for example, is executed to determine whether or not imaging using the wireless RF coil unit 18 can be performed without problems by estimating power consumption of the battery provided in the wireless RF coil unit 18 on the basis of the imaging conditions when imaging of the subject P is performed and the battery information of the battery provided in the wireless RF coil unit 18 and to present (show) a determination result or a proposal based on the determination result to the performer of the MRI examination.

### [Imaging Procedure Based on MRI Apparatus]

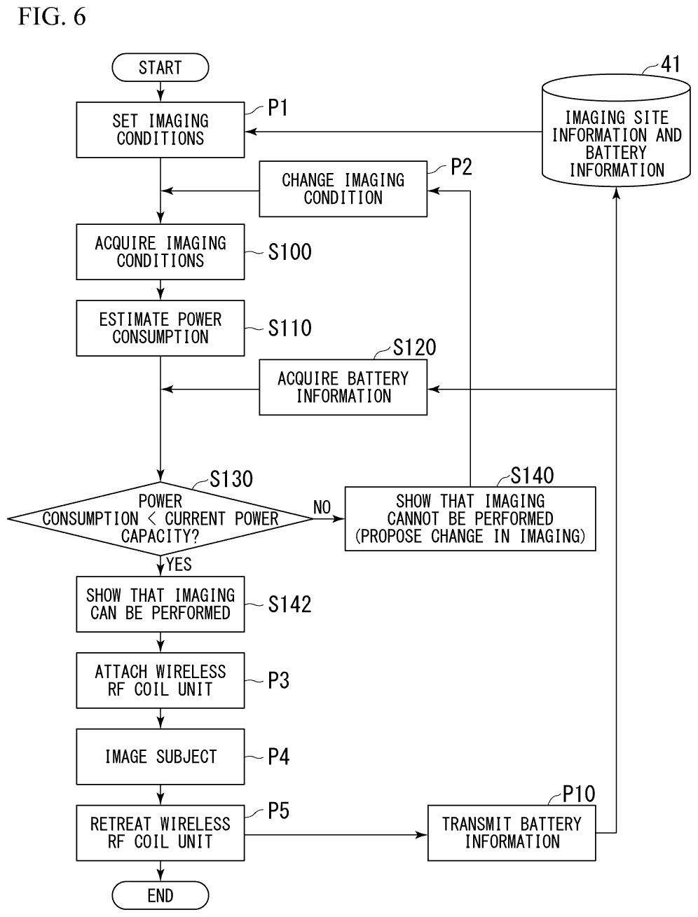

Next, a procedure for imaging the subject P in the MRI apparatus 1 and a flow of a process of the coil determination function 51 will be described. FIG. 6 is a flowchart showing an example of the procedure for imaging the subject in the medical image diagnosis apparatus (the MRI apparatus 1) including the medical information processing device (the coil determination function 51) according to the embodiment and the flow of the process of the medical information processing device (the coil determination function 51). The flowchart shown in FIG. 6 is an example of the case where the subject P is imaged using the wireless RF coil unit 18 in the MRI apparatus 1. In the following description, it is assumed that imaging site information (a series of sequence information items corresponding to the same imaging site) is stored in the memory 41. Furthermore, it is assumed that the memory 41 stores information about the capacity of power currently stored in the battery provided in the wireless RF coil unit 18 (current battery information), and that the stored battery information is updated every time new battery information is transmitted from the wireless RF coil unit 18 or the charger.

In procedure P1, the performer of the MRI examination sets the imaging conditions for imaging the subject P. For example, the performer of the MRI examination sets the imaging conditions (setting values) using the setting image IM1 shown in FIG. 4 and selects (designates) the wireless RF coil units 18 and the coil elements constituting the wireless RF coil units 18 using the setting image IM2 shown in FIG. 5. The performer of the MRI examination may be executed to set imaging site information corresponding to the imaging site of the subject P within the imaging site information stored in the memory 41 as the imaging conditions.

When the performer of the MRI examination sets the imaging conditions for imaging the subject P, the imaging condition acquisition function 511 executed in the coil determination function 51 is executed to acquire the set imaging conditions and outputs the acquired imaging conditions to the power consumption estimation function 512 as imaging condition information (step S100).

The power consumption estimation function 512 executed in the coil determination function 51 is executed to estimate the power consumption of the battery provided in the wireless RF coil unit 18 for use in imaging on the basis of the imaging condition information output by the imaging condition acquisition function 511 and output the estimated power consumption information to the imaging determination function 514 (step S110).

The battery information acquisition function 513 executed in the coil determination function 51 is executed to acquire the current battery information of the battery provided in the selected (designated) wireless RF coil unit 18 included in the imaging condition information (or read the current battery information from the memory 41) and output the current battery information to the imaging determination function 514 (step S120).

The imaging determination function 514 executed in the coil determination function 51 is executed to compare the power consumption information output by the power consumption estimation function 512 with the battery information output by the battery information acquisition function 513, and ascertain whether or not the estimated power consumption is less than the current power capacity (step S130). When the estimated power consumption is greater than or equal to the current power capacity in step S130, the imaging determination function 514 determines that imaging using the wireless RF coil unit 18 cannot be performed. The imaging determination function 514 is executed to output this determination result to the determination result presentation function 515.

The determination result presentation function 515 executed in the coil determination function 51 is executed to present (show) the determination result (indicating that imaging cannot be performed) output by the imaging determination function 514 to the performer of the MRI examination (step S140). At this time, the determination result presentation function 515 is executed to propose a change in the imaging using the wireless RF coil unit 18 to the performer of the MRI examination.

In procedure P2, the performer of the MRI examination changes the imaging conditions for imaging the subject P in accordance with the proposal. Thereby, the coil determination function 51 is executed to iterate the processing of steps S100 to S130 to iteratively determine whether or not imaging using the wireless RF coil unit 18 can be performed for the current imaging condition settings.

On the other hand, when the estimated power consumption is less than the current power capacity in step S130, the imaging determination function 514 is executed to determine that imaging using the wireless RF coil unit 18 can be performed. The imaging determination function 514 is executed to output this determination result to the determination result presentation function 515. The determination result presentation function 515 is executed to present (show) the determination result (indicating that imaging can be performed) output by the imaging determination function 514 to the performer of the MRI examination (step S142).

In procedure P3, the performer of the MRI examination attaches the selected (designated) wireless RF coil unit 18 to the subject P. Also, in procedure P4, the performer of the MRI examination performs imaging of the subject P. Thereby, in the MRI apparatus 1, an MR image of the subject P is displayed on the display 42 and the performer of the MRI examination can ascertain the MR image of the subject P. When imaging of the subject P is completed, the performer of the MRI examination retracts the wireless RF coil unit 18 attached to the subject P in procedure P5, and ends the procedure for imaging the subject P in the MRI apparatus 1. At this time or thereafter, in procedure P10, when the wireless RF coil unit 18 or a charger that charges the wireless RF coil unit 18 transmits current battery information, the battery information acquisition function 513 is executed to acquire the transmitted battery information and cause the memory 41 to store the acquired battery information.

As described above, the coil determination function 51, which is the medical information processing device of the embodiment, is executed to determine whether or not imaging using the wireless RF coil unit 18 can be performed under the current imaging condition settings when the subject P is imaged using the wireless RF coil unit 18 in the MRI apparatus 1 and presents (shows) the determination result and the proposal based on the determination result to the performer of the MRI examination. Thereby, in the MRI apparatus 1 to which the medical information processing device of the embodiment is applied, imaging of the subject P can be performed while the power storage state of the battery provided in the wireless RF coil unit 18 is appropriately managed. Thereby, in the MRI apparatus 1 to which the medical information processing device of the embodiment is applied, the performer of the MRI examination does not need to take into consideration the power storage state of the battery provided in the wireless RF coil unit 18, and imaging using the wireless RF coil unit 18 can be efficiently performed.

The case where the memory 41 of the MRI apparatus 1 to which the medical information processing device (the coil determination function 51) of the embodiment is applied stores the acquired battery information and the stored battery information corresponding to the same battery is updated to constantly update the current battery information to the latest information has been described in the above-described embodiment. However, the present invention is not limited to the case where the acquired battery information is used as the current battery information acquired when imaging using the wireless RF coil unit 18 is subsequently performed by constantly updating the battery information to the latest information. For example, instead of updating the battery information stored in the memory 41, the battery information may be associated with acquisition times, saved as a history of changes in the battery information, and stored in a database. In this case, an external memory with which the MRI apparatus 1 can communicate is conceivable as a saving destination of the battery information stored in the database. The battery information stored in the database may be reflected in the estimation of the power consumption of the battery in the power consumption estimation function 512 or may be used when the deterioration state of the battery is determined. For example, when the power consumption of the battery is estimated in the power consumption estimation function 512, because information about the actual power consumption of the battery can be obtained on the basis of pre- and post-imaging battery information stored in the database, the estimation when similar imaging is performed can be performed more accurate. For example, because imaging is performed under the same imaging conditions for a subject P undergoing follow-up observation, it is possible to estimate battery power consumption close to the actual battery power consumption on the basis of battery information stored in the database during previous imaging. For example, the deterioration state of the battery can be determined on the basis of a relationship between the iteration of charging and discharging of the battery indicated in the battery information stored in the database and the power capacity at that time.

In the above-described embodiment, the case where the performer of the MRI examination sets the imaging conditions for imaging the subject P in procedure P1 and then the medical information processing device (the coil determination function 51) of the embodiment determines whether or not imaging using the wireless RF coil unit 18 can be performed in the setting of the imaging conditions and performs imaging of the subject P when it is determined that imaging can be performed has been described. That is, a case where an imaging condition setting process and an imaging process are performed at the same time has been described in the above-described embodiment. This, for example, is equivalent to a case where imaging of the subject P (i.e., an examination of the subject P) is urgently performed in a medical institution. However, it is easily assumed that a reservation is first made in the medical institution and then imaging of the subject P is performed on another day. In this case, the procedure of the performer of the MRI examination and the flow of the process in the coil determination function 51 can be easily conceived on the basis of an example of the procedure of the performer of the MRI examination and the flow of the process in the coil determination function 51 shown in FIG. 6. More specifically, the setting of the imaging conditions in procedure P1 shown in FIG. 6 is set as the procedure of the performer of the MRI examination, which sets the imaging conditions when a reservation is made, registers the subject information, and sets the imaging site information. In this procedure, even if the information (setting value) of the imaging timing or the selection (designation) of the RF coil or coil element for use in imaging is not set in the setting of the imaging conditions, for example, it is possible to obtain the information (setting value) of the imaging timing and the information of the selected (designated) RF coil or coil element on the basis of a series of sequence information items indicated in the set imaging site information or registered subject information (e.g., information about a height). Also, the coil determination function 51 is executed to perform the processing of steps S100 to S142 shown in FIG. 6 on the basis of the information obtained in the procedure when a reservation is made. At this time, in addition to the determination of whether or not imaging can be performed for the setting of the imaging conditions of the subject making the reservation this time, the coil determination function 51 may also be executed to determine whether or not imaging including the setting of the imaging conditions of another subject already reserved on the same day can be performed. Meanwhile, the battery information (currently stored power capacity) of the battery provided in the wireless RF coil unit 18 in the reservation step is different from the battery information on the day when imaging is performed. Therefore, in the reservation step, the coil determination function 51 (more specifically, the imaging determination function 514) is executed to determine whether or not imaging can be performed under the assumption that the batteries of all the wireless RF coil units 18 are fully charged, as described above. However, considering that imaging of multiple subjects is reserved on the same day, it is considered very useful to determine in advance whether or not imaging using the wireless RF coil units 18 can be performed with the setting of the imaging conditions corresponding to each subject. Moreover, as in the above-described proposal 5, the coil determination function 51 (more specifically, the determination result presentation function 515) can be executed to make a proposal to the performer of the MRI examination so that the determination (consideration) including the replacement with the sequential order of another subject to be imaged on the same day (rearrangement of the imaging plan) or the change in the reservation date is performed in accordance with the determination result. In this way, in the MRI apparatus 1 to which the medical information processing device (the coil determination function 51) of the embodiment is applied, it is possible to determine in advance whether or not imaging using the wireless RF coil units 18 can be performed in the reservation step. Also, on the day of imaging, the final determination of whether or not imaging using the wireless RF coil unit 18 can be performed and the actual imaging can be performed using the procedure of the performer of the MRI examination shown in FIG. 6 and the processing flow of the coil determination function 51.

The above-described embodiment can be expressed as follows.

A medical information processing device for managing a power storage state of a battery provided in an RF coil unit to be mounted on a subject to be scanned by a magnetic resonance imaging apparatus, the RF coil unit being configured to receive a magnetic resonance signal as an analog data from the subject generated by applying a radio frequency (RF) pulse to the subject, to convert the magnetic resonance signal into a magnetic resonance data as a digital data, and to transmit the magnetic resonance data by a wireless communication, the medical information processing device including: processing circuitry,
wherein the processing circuitry
acquires an imaging condition to be used when the subject is scanned by the magnetic resonance imaging apparatus,
estimates, based on the imaging condition, an amount of power of the battery of the RF coil unit consumed by a scanning of the subject on the basis of the imaging condition by the magnetic resonance imaging apparatus,
acquires battery information including an amount of the power stored in the battery, and
determines, based on the estimated amount of the power and the acquired battery information, whether or not the RF coil unit can be used to scan the subject based on the imaging condition by the magnetic resonance imaging apparatus.

According to at least one embodiment described above, there is provided a medical information processing device (51) for managing a power storage state of a battery provided in an RF coil unit (18) to be mounted on a subject (P) to be scanned by a magnetic resonance imaging apparatus (1), the RF coil unit being configured to receive a magnetic resonance signal as an analog data from the subject generated by applying a radio frequency (RF) pulse to the subject, to convert the magnetic resonance signal into a magnetic resonance data as a digital data, and to transmit the magnetic resonance data by a wireless communication, the medical information processing device (51) including: processing circuitry configured to acquire an imaging condition to be used when the subject is scanned by the magnetic resonance imaging apparatus (511), estimate, based on the imaging condition, an amount of power of the battery of the RF coil unit consumed by a scanning of the subject based on the imaging condition by the magnetic resonance imaging apparatus (512), acquire battery information including an amount of the power stored in the battery (513), and determine, based on the estimated amount of the power and the acquired battery information, whether or not the RF coil unit can be used to scan the subject based on the imaging condition by the magnetic resonance imaging apparatus (514), whereby the power storage state of the battery provided in the wireless RF coil unit (18) can be preferably managed when the RF coil (16) of the magnetic resonance imaging apparatus (1) is wirelessly configured.

While certain embodiments of the present invention have been described, these embodiments have been presented by way of example only, and are not intended to limit the scope of the inventions. These embodiments may be embodied in a variety of other forms. Various omissions, substitutions, and modifications may be made without departing from the spirit of the inventions. The inventions described in the accompanying claims and their equivalents are intended to cover such embodiments or modified examples as would fall within the scope and spirit of the inventions.

What is claimed is:

1. A medical information processing device for managing a power storage state of a battery provided in a radio frequency (RF) coil unit to be mounted on a subject to be scanned by a magnetic resonance imaging apparatus, the RF coil unit being configured to receive a magnetic resonance signal as an analog data from the subject generated by applying a radio frequency (RF) pulse to the subject, to convert the magnetic resonance signal into a magnetic resonance data as a digital data, and to transmit the magnetic resonance data by a wireless communication, the medical information processing device comprising:
processing circuitry configured to:
acquire an imaging condition to be used when the subject is scanned by the magnetic resonance imaging apparatus,
estimate, based on the imaging condition, an amount of power of the battery of the RF coil unit consumed by a scanning of the subject based on the imaging condition by the magnetic resonance imaging apparatus,
acquire battery information including an amount of the power stored in the battery, and
determine, based on the estimated amount of the power and the acquired battery information, whether or not the RF coil unit can be used to scan the subject based on the imaging condition by the magnetic resonance imaging apparatus.

2. The medical information processing device according to claim 1,
wherein the processing circuitry is configured to determine that the RF coil unit can be used to scan the subject based on the imaging condition by the magnetic resonance imaging apparatus in a case where the amount of the power stored in the battery is greater than the estimated amount of the power consumed by the scanning of the subject based on the imaging condition by the magnetic resonance imaging apparatus; and
determine that the RF coil unit can not be used to scan the subject based on the imaging condition by the magnetic resonance imaging apparatus in a case where the amount of the power stored in the battery is less than the estimated amount of the power consumed by the scanning of the subject based on the imaging condition by the magnetic resonance imaging apparatus.

3. The medical information processing device according to claim 1,
wherein the processing circuitry is configured to change the imaging condition as a first imaging condition to a second imaging condition in a case where the RF coil unit can not be used to scan the subject based on the first imaging condition by the magnetic resonance imaging apparatus.

4. The medical information processing device according to claim 1,
wherein the processing circuitry is configured to display a result of the determining whether or not the RF coil unit can be used to scan the subject based on the imaging condition by the magnetic resonance imaging apparatus on a display.

5. The medical information processing device according to claim 1,
wherein the imaging condition includes information indicating a reception time during which the RF coil unit receives the magnetic resonance signal from the subject,
wherein the estimated amount of power of the battery of the RF coil unit consumed by the scanning of the subject based on the imaging condition by the magnetic resonance imaging apparatus includes an amount of power consumed during the reception time.

6. The medical information processing device according to claim 1,
wherein the RF coil unit includes a plurality of coil elements,
wherein the imaging condition includes information indicating a designating the coil element to be used for receiving the magnetic resonance signal from the subject, and
wherein the estimated amount of power of the battery of the RF coil unit consumed by the scanning of the subject based on the imaging condition by the magnetic resonance imaging apparatus includes an amount of power consumed by the designated coil elements during receiving the magnetic resonance signal.

7. A medical information processing method comprising:
acquiring, by a computer of a medical information processing device for managing a power storage state of a battery provided in an RF coil unit to be mounted on a subject to be scanned by a magnetic resonance imaging apparatus, the RF coil unit being configured to receive a magnetic resonance signal as an analog data from the subject generated by applying a radio frequency (RF) pulse to the subject, to convert the magnetic resonance signal into a magnetic resonance data as a digital data, and to transmit the magnetic resonance data by a wireless communication, an imaging condition to be used when the subject is scanned by the magnetic resonance imaging apparatus,
estimating, by the computer, based on the imaging condition, an amount of power of the battery of the RF coil unit consumed by a scanning of the subject based on the imaging condition by the magnetic resonance imaging apparatus,
acquiring, by the computer, battery information including an amount of the power stored in the battery, and
determining, by the computer, based on the estimated amount of the power and the acquired battery information, whether or not the RF coil unit can be used to scan the subject based on the imaging condition by the magnetic resonance imaging apparatus.

8. A magnetic resonance imaging apparatus, comprising:
a magnet configured to generate a magnetic field;
a top plate on which a subject to be scanned is placed; and
a medical information processing device for managing a power storage state of a battery provided in a radio frequency (RF) coil unit to be mounted on the subject, the RF coil unit being configured to receive a magnetic resonance signal as an analog data from the subject generated by applying a radio frequency (RF) pulse to the subject, to convert the magnetic resonance signal into a magnetic resonance data as a digital data, and to transmit the magnetic resonance data by a wireless communication, the medical information processing device including a processing circuitry configured to:
acquire an imaging condition to be used when the subject is scanned by the magnetic resonance imaging apparatus,
estimate, based on the imaging condition, an amount of power of the battery of the RF coil unit consumed by a scanning of the subject based on the imaging condition by the magnetic resonance imaging apparatus,
acquire battery information including an amount of the power stored in the battery, and
determine, based on the estimated amount of the power and the acquired battery information, whether or not the RF coil unit can be used to scan the subject based on the imaging condition by the magnetic resonance imaging apparatus.
